(19) United States
(12) Patent Application Publication
(10) Pub. No.: US 2026/0155260 A1
(43) Pub. Date: Jun. 4, 2026

(54) GROWTH AGE ESTIMATION METHOD USING ARTIFICIAL INTELLIGENCE MODEL

(71) Applicant: GP CO., LTD., Gwangmyeong-si (KR)

(72) Inventors: Je Hyeok SEONG, Gwangmyeong-si Gyeonggi-do (KR); Ji Hun KIM, Gwacheon-si (KR); Do Hyun CHUN, Seoul (KR)

(21) Appl. No.: 19/459,407

(22) Filed: Jan. 26, 2026

Related U.S. Application Data
(63) Continuation of application No. PCT/KR2024/097095, filed on Dec. 18, 2024.

(30) Foreign Application Priority Data
Dec. 20, 2023 (KR) 10-2023-0187475

(51) Int. Cl. G16H 50/30; A61B 5/00

(52) U.S. Cl. CPC G16H 50/30; A61B 5/48

(57) ABSTRACT

A growth age estimation method using an artificial intelligence model, that may include the steps of: receiving biometric data of a subject to be measured; using the biometric data of the subject to be measured, thereby extracting predicted age of peak height velocity (APHV) data, which is the expected age of maximum growth rate; and, on the basis of the predicted APHV and second APHV data, which is the average value of first APHV data, calculating the growth age of the subject to be measured.

### TECHNICAL FIELD

The present invention relates to a method of estimating a biological growth age, rather than an actual chronological age, based on age of peak height velocity (APHV) data representing an age at which a growth velocity extracted from biometric data of growing children and adolescents reaches a maximum value, and providing a customized solution for each growth stage.

### BACKGROUND ART

Recently, the development of artificial intelligence technology has led to its application in various fields, and methods for extracting inherent features from data through a neural network model instead of existing data processing schemes to generate additional information have been developed and used.

A neural network model used for artificial intelligence can detect and recognize features from input data through learning more quickly and accurately than general data processing. Recently, artificial intelligence technology is being applied not only to simple tracking and detection of objects but also to learning past histories and deriving current features that reflect future predictions or time-series changes.

Here, predictive analysis is a technology in the field of statistics and data mining for extracting information from data and using the information to predict trends, behavioral patterns, and the like. Such predictive analysis may be applied to all fields necessary for decision making based on the information obtained from the data. The core of predictive analysis lies in understanding relationships between variables and then predicting unknown variables.

To this end, various approaches are used depending on data characteristics and prediction targets.

Various fields in which predictive analysis is required include the field of physical growth of adolescents. There is great interest among parents, adolescents, and the like in when growth in height occurs and how much growth will take place.

For conventional prediction of the growth in height, a method of predicting the growth in height using an X-ray of growth plates or analyzing relationships with genetic or environmental factors has been presented (Patent Nos. 10-2075743 and 10-1866208), and a method of transforming physical data of sample subjects, whose measurement times or numbers of measurements differ, into a form suitable for training of a growth prediction model has been presented (Patent No. 10-2198302).

Although methods for predicting physical growth of children and adolescents have been presented in the related art, research on methods for predicting the risk of occurrence of precocious puberty, obesity, and the like that may accompany the growth of children and adolescents as described above and providing corresponding solutions has been insufficient.

Further, since children and adolescents have growth stages having different characteristics, this should be considered to improve the reliability of predicted data and analysis-based solution provision.

Meanwhile, in the related art disclosed so far, various methods have been disclosed in which physical data or biometric data of children and adolescents is directly input to a defined growth prediction tool to predict growth, but according to such related art, there is a disadvantage that it is difficult to provide a personalized solution because growth is predicted only based on general indicators such as past and current heights, weights, and BMIs without considering individual growth characteristics such as a growth velocity of children and adolescents, and whether a growth stage is reached earlier compared to other sample subjects.

Also, although a superimposition by translation and rotation (SITAR) methodology has emerged in relation to a height growth velocity, there is a problem that measurement values over an entire growth period are required to estimate a growth velocity curve using the SITAR methodology, making only post-hoc estimation possible.

Meanwhile, a conventional height growth prediction model is a model for predicting a height growth curve using an LGBM model with a height, weight, body composition, their quantiles, and a benchmark growth rate based on a current quantile as input variables, and upward bias in an estimation model is likely to occur in a specific dataset in which, at older ages, a height quantile is high and a proportion of observed values is high. Further, although the conventional height growth prediction model has 52 variables related to body composition, the conventional height growth prediction model utilizes only eight variables including height, weight, and body composition mass, and therefore has a disadvantage in that the prediction accuracy of the estimation model is low.

As a result, in the conventional height growth prediction model, although a growth velocity trend of the sample subject is important for height prediction, information on past measurement values is not reflected in the estimation model, and therefore, there is a need to measure a tracking growth velocity reflecting the past information.

Further, the importance of a method of estimating a biological growth age rather than an actual age, that is, an actual chronological age uniformly calculated from the time of birth, using such a tracking growth rate, that is, estimating an age indicating how old a body biologically corresponds to, based on a growth state, is emerging.

### DISCLOSURE

### Technical Problem

The present invention is directed to providing a method, device, and computer program for classifying growth stages based on an age of peak height velocity (APHV) data representing an age at which a growth velocity of growing children and adolescents reaches a maximum value, predicting growth of a height, obesity, disease, and the like, and providing a customized solution for each growth stage.

The present invention is also directed to providing a method of estimating tracking growth velocity reflecting past information of children and adolescents using an APHV estimation model constructed through artificial intelligence and estimating a biological growth age indicating how old a body biologically corresponds to, based on a growth state of the children and adolescents.

The present invention is also directed to providing a method, device, and computer program for predicting growth of a height, obesity, disease, and the like based on growth stages and providing a customized solution for each growth stage.

The present invention is also directed to providing a method, device, and computer program for predicting growth of a height, obesity, disease, and the like based on growth stages of children and adolescents using an APHV estimation model constructed through artificial intelligence, and providing a customized solution for each growth stage.

### Technical Solution

According to an aspect of the present invention, there is provided a growth age estimation method using an artificial intelligence model trained with first age of peak height velocity (APHV) data representing an age at which a growth velocity of a plurality of sample subjects reaches a maximum value, as target data, the method including receiving biometric data of a measurement subject; extracting predicted APHV data representing an age at which the growth velocity is expected to reach a maximum value using the biometric data of the measurement subject; and calculating a growth age of the measurement subject based on second APHV data that is an average value of the first APHV data, and the predicted APHV.

The calculating of the growth age is performed using Equation 1:

$${GA}_{i}^{′} = ( {CA}_{i} - {APHV}_{i}^{′} ) + {APHV}_{G} \qquad [ Equation 1 ]$$

where, GAᵢ′ may denote an estimated growth age of the measurement subject, CAᵢ may denote a chronological age/calendar age of the measurement subject, APHVᵢ′ may denote the predicted APHV data including the estimated and predicted APHV values of the measurement subject, and APHV_{G} may denote the second APHV data which is a representative value or an average value of the first APHV data derived from a training data group.

The extracting of the predicted APHV data may include estimating an expected profile of the growth velocity y using the biometric data of the measurement subject; and extracting a predicted age of onset of growth spurt (AOGS) representing an age at which an increase in the growth velocity of the measurement subject begins, and predicted APHV representing an age at which the growth velocity of the measurement subject has a maximum value.

The biometric data of the measurement subject may include at least two or more pieces of time-series biometric data measured at different ages.

The first APHV data may be a set of data obtained by receiving biometric data of the plurality of sample subjects, estimating a plurality of first growth velocity curves showing a velocity of height growth with respect to actual age for each of the plurality of sample subjects, and extracting an age at which the growth velocity reaches a maximum value for each of the plurality of first growth velocity curves.

According to another aspect of the present invention, there is provided a growth age estimation method using an artificial intelligence model trained with parameters and feature points for determining a plurality of first growth velocity curves showing a velocity of height growth with respect to actual age of a plurality of sample subjects as target data, the method including receiving biometric data of a measurement subject; extracting predicted age of peak height velocity (APHV) data representing an age at which the growth velocity is expected to reach a maximum value using the biometric data of the measurement subject; and calculating the growth age of the measurement subject based on the predicted APHV and second APHV data that is an average value of first APHV data representing an age at which a growth velocity of the plurality of sample subjects reaches a maximum value among feature points determining the plurality of first growth velocity curves.

The calculating of the growth age may be performed using Equation 1:

$${GA}_{i}^{′} = ( {CA}_{i} - {APHV}_{i}^{′} ) + {APHV}_{G} \qquad [ Equation 1 ]$$

where GAᵢ′ may denote an estimated growth age of the measurement subject, CAᵢ may denote a chronological age/calendar age of the measurement subject, APHVᵢ′ may denote the predicted APHV data including the estimated and predicted APHV values of the measurement subject, and APHV_{G} may denote the second APHV data which is a representative value or an average value of the first APHV data derived from a training data group.

The biometric data of the measurement subject may include at least two or more pieces of time-series biometric data measured at different ages.

The first APHV data may be a set of data obtained by extracting an age at which the growth velocity reaches a maximum value for a first growth velocity curve of each of the plurality of sample subjects.

The first growth velocity curve may be estimated by extracting age data and height data among the time-series biometric data of the sample subjects and then applying the age data and the height data to a curve transformation model with the age data and the height data as input variables.

### Advantageous Effects

The growth age estimation model based on biometric data using artificial intelligence according to exemplary embodiments of the present invention applies a tracking growth velocity extracted from past information to a statistical methodology to estimate a growth velocity curve, extracts an age of peak height velocity (APHV) data representing an age at which the growth velocity reaches its maximum with respect to the growth velocity curves, and then utilizes the APHV data for construction of various prediction models such as an integrated height growth prediction model, a precocious puberty prediction model, and an obesity prediction model, thereby maximizing the reliability of various prediction models such as the integrated height growth prediction model, the precocious puberty prediction model, and the obesity prediction model.

Further, since a biological growth age rather than an actual age, that is, a actual chronological age uniformly calculated from the time of birth, is estimated using the tracking growth velocity, that is, an age indicating how old a body biologically corresponds to is estimated based on a growth state, and the biological growth age is utilized for construction of various prediction models such as an integrated height growth prediction model, a precocious puberty prediction model, and an obesity prediction model, it is possible to maximize the reliability of various prediction models such as the integrated height growth prediction model, the precocious puberty prediction model, and the obesity prediction model.

Further, according to various embodiments of the present invention, it is possible to accurately predict the heights of children and adolescents in consideration of the growth stages of children and adolescents using the growth age estimation model constructed through artificial intelligence learning, and to provide a solution necessary for growth in height in consideration of each growth stage.

### DESCRIPTION OF DRAWINGS

FIG. 1 is a configuration diagram illustrating a configuration of a growth age estimation model based on biometric data using artificial intelligence according to exemplary embodiments of the present invention.

FIG. 2 is a diagram illustrating a predicted height and target height for each growth stage according to exemplary embodiments of the present invention.

FIG. 3 is a flowchart illustrating a growth age estimation method based on biometric data using artificial intelligence according to exemplary embodiments of the present invention.

FIGS. 4A-4B are diagrams illustrating a plurality of first growth velocity curves showing a velocity of height growth with respect to actual age using time-series biometric data of sample subjects.

FIGS. 5 and 6 are diagrams illustrating APHV estimation errors according to age in months for girls and boys, respectively.

FIGS. 7 and 8 are diagrams illustrating the importance of APHV prediction variables for females and males, respectively.

FIGS. 9A-9B are diagrams illustrating variable importance in conventional female and male growth prediction models, and FIGS. 10A-10B are diagrams illustrating variable importance in a growth prediction model of the present invention.

### MODES OF THE INVENTION

Hereinafter, specific embodiments of the present invention will be described. The following detailed description is provided to facilitate a comprehensive understanding of the methods, devices, and/or systems described in the present specification. However, this is merely an example, and the present invention is not limited thereto.

When it is determined that detailed description of known technologies related to the present invention may unnecessarily obscure the gist of the present invention in describing the embodiments of the present invention, the detailed description will be omitted. The terms to be described below are terms defined in consideration of functions in the present invention and may vary depending on, for example, a user's or operator's intention or practice. Therefore, the definitions should be made based on the overall content of the present specification. The terms used in the detailed description are merely for describing embodiments of the present invention and should not be construed as limiting. Unless clearly used otherwise, expressions in singular forms include plural forms. In the present description, expressions such as “including” or “comprising” are intended to indicate certain features, numerals, steps, operations, elements, some thereof, or combinations thereof, and should not be construed to exclude the presence or possibility of one or more other features, numerals, steps, operations, elements, some thereof, or combinations thereof in addition to those described.

Terms such as first, second, A, B, (a), and (b) may be used in describing components of the embodiments of the present invention. These terms are only for distinguishing the components from each other and do not limit the nature, order, or sequence of the components.

In an exemplary embodiment of the present invention, children and adolescents may be understood as concepts including a period during which a human body grows. More specifically, the infants, children, and adolescents that refer to sample subjects in the exemplary embodiment of the present invention are defined hereinafter through the meanings of baby, child, adolescent, toddler, and infant.

The infant is a person in a period continuing from a neonatal period and is a period up to two years after birth during which a person grows while holding a mother's nipple in the mouth, and infancy is a period in which all experiences of nutrition, caress, and excretion influence subsequent general tendencies, and the child is a term that usually refers to a person belonging to an age group of 6 years or older and under 13 and may also include a toddler (aged from 1 to 5 years) in a broad sense.

The adolescent is a person in an intermediate period between childhood and adulthood and generally refers to a person aged 13 years or older and under 19 years. The toddler may refer to a person from one year after birth to age 5. The child may generally refer to a person up to age 15.

Therefore, the children and adolescents that are the sample subjects may, in a narrow sense, refer to persons in a period from age 5 to age 19, including children and adolescents, and may indicate a period including all general periods in which physical growth occurs, including from infancy to adolescence, in a broad sense.

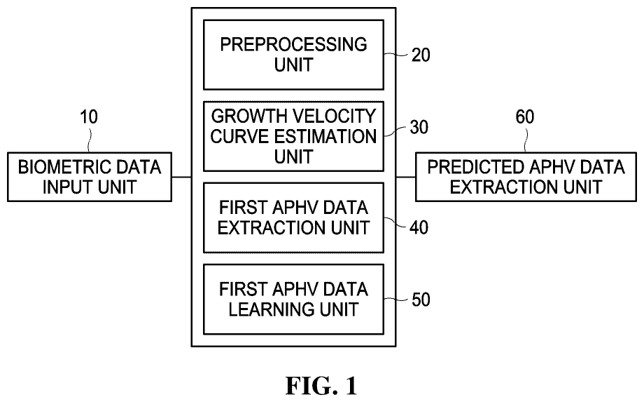

FIG. 1 is a configuration diagram illustrating a configuration of a growth age estimation model based on biometric data using artificial intelligence according to exemplary embodiments of the present invention.

Referring to FIG. 1, the growth age estimation model based on biometric data using artificial intelligence according to exemplary embodiments of the present invention may include a biometric data input unit 10, a preprocessing unit 20, a growth velocity curve estimation unit 30, a first APHV data extraction unit 40, and a first APHV data learning unit 50, a predicted APHV data extraction unit 60, a second APHV data extraction unit 70, and a growth age calculation unit 80.

The growth age estimation model based on biometric data using artificial intelligence according to exemplary embodiments of the present invention may receive time-series physical information for a sample subject through the biometric data input unit 10. The physical information of the sample subject may include not only basic information such as school grade (or age), sex, and height, but also additional information such as weight, protein, mineral content, body fat, body water, muscle mass (soft lean mass), fat-free mass, bone tissue, skeletal muscle mass, body mass index (BMI), basal metabolic rate, neck circumference, chest circumference, abdominal circumference, thigh circumference, arm circumference, and hip circumference. Such physical information is merely one example to aid understanding of the present invention, and the present embodiment is not limited thereto, and it is obvious that the types of information constituting the physical information may vary according to the embodiment.

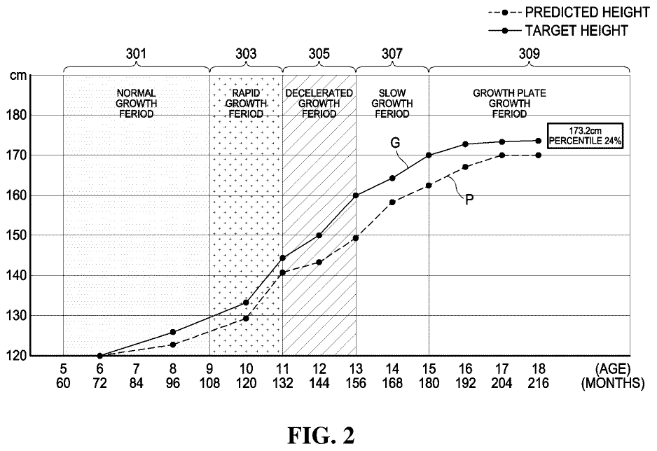

The time-series physical information of the sample subject may be continuous information or discontinuous information but may be information included in at least one of the periods corresponding to the growth stages in FIG. 2.

More specifically, the time-series physical information of the sample subject may be collected at various collection timings and numbers of collections. For example, in the case of a first sample subject, there may be physical information measured during 8 to 12 years of age which is a portion of childhood and adolescence, and in the case of a second sample subject, there may be physical information measured irregularly such as at ages 8, 10 to 12, and 15. Further, in the case of a third sample subject, there may be physical information measured several times within a certain period (within a period of any one growth stage among the plurality of growth stages in FIG. 2), whereas in the case of a fourth sample subject, there may be physical information measured only once within a certain period (within a period of any one growth stage among the plurality of growth stages in FIG. 2).

As described above, the physical information of the sample subject may be included in two or more of the growth stages in FIG. 2 depending on the collection timings and numbers of collections (the first sample subject and the second sample subject), but there may also be cases in which that is not the case (the third sample subject and the fourth sample subject).

The preprocessing unit 20 may be controlled to perform preprocessing on the time-series biometric data of the sample subjects.

In exemplary embodiments, the preprocessing unit 20 may perform preprocessing by determining all pieces of biometric data of a sample subject without biometric data collected during a specific growth period to be noise and removing the biometric data. This is intended to improve the prediction accuracy of the APHV estimation model based on biometric data according to the present invention and may not be applied in a state in which data is sufficiently accumulated, and in that case, preprocessing may be performed by supplementing or additionally generating some biometric data through various methods such as an artificial intelligence model and big data analysis, instead of determining all the pieces of biometric data of the sample subject without biometric data collected during the specific growth period to be noise and removing the biometric data.

Meanwhile, the biometric data input unit 10 may be further controlled to receive physical information of a measurement subject for whom APHV estimation is desired, in addition to receiving the time-series physical information of the sample subject. In this case, the physical information of the sample subject may include not only basic information such as school grade (or age), sex, and height, but also additional information such as weight, protein, mineral content, body fat, body water, muscle mass (soft lean mass), fat-free mass, bone tissue, skeletal muscle mass, body mass index (BMI), basal metabolic rate, neck circumference, chest circumference, abdominal circumference, thigh circumference, arm circumference, and hip circumference.

The growth velocity curve estimation unit 30 may be controlled to estimate a plurality of first growth velocity curves showing a velocity of height growth with respect to actual age of each of the plurality of sample subjects using the time-series biometric data of the sample subjects.

In exemplary embodiments, the estimation of the first growth velocity curve may be performed by extracting age data and height data from the time-series biometric data of the sample subjects and then applying the age data and the height data to a curve transformation model as input variables.

In this case, the type of curve transformation model is not particularly limited. That is, the curve transformation model may be a model constructed using a polynomial function, and for example, the curve transformation model may be a model constructed using Equation 2.

$${y}_{it} = {α}_{i} + h ( \frac{t - {β}_{i}}{exp ( - {γ}_{i} )} ) \qquad [ Equation 2 ]$$

where yᵢₜ is, αᵢ, βᵢ, and γᵢ are random correction values for adjusting the vertical shift, horizontal shift, and slope of the curve respectively, h may be a specific function related to the height data, and t may be the age data.

However, the concept of the present invention is not necessarily limited thereto, and may be a model constructed using a polynomial function other than Equation 2.

The first APHV data extraction unit 40 may be controlled to extract the first APHV data representing an age at which the growth velocity reaches a maximum value for each of the plurality of first growth velocity curves.

The first APHV data may be utilized for growth stage diagnosis, construction of the estimation model, and derivation of a growth age, and the growth age and the tracking growth velocity may ultimately be utilized for construction of various prediction models such as an integrated height growth prediction model, a precocious puberty prediction model, and an obesity prediction model.

Meanwhile, the conventional height growth prediction model is a model for predicting a height growth curve using an LGBM model with a height, weight, body composition, their quantiles, and a benchmark growth rate based on a current quantile as input variables, and upward bias in an estimation model is likely to occur in a specific dataset in which, at older ages, a height quantile is high and a proportion of observed values is high. Further, although the conventional height growth prediction model has 52 variables related to body composition, the conventional height growth prediction model utilizes only eight variables including height, weight, and body composition mass, and therefore has a disadvantage in that the prediction accuracy of the estimation model is low.

That is, in the conventional height growth prediction model, although a growth velocity trend of the sample subject is important for height prediction, information on past measurement values is not reflected in the estimation model, and therefore, there is a need to measure a tracking growth velocity reflecting the past information. The prevent invention is characterized in that the APHV estimation model based on biometric data using artificial intelligence according to exemplary embodiments of the present invention applies a tracking growth velocity extracted from past information to a statistical methodology to estimate a growth velocity curve, extracts APHV data representing an age at which the growth velocity reaches its maximum with respect to the growth velocity curves, and then utilizes the APHV data for construction of various prediction models such as an integrated height growth prediction model, a precocious puberty prediction model, and an obesity prediction model.

The first APHV data learning unit 50 may be controlled to train an artificial intelligence model by inputting the first APHV data to the artificial intelligence model as target data.

The predicted APHV data extraction unit 60 may be controlled to extract predicted APHV data representing an age at which the growth velocity of the measurement subject is expected to become a maximum, by applying the biometric data of the measurement subject to the artificial intelligence model trained using the first APHV data representing an age at which a growth velocity of the plurality of sample subjects reaches a maximum value as the target data.

The second APHV data extraction unit 70 may be controlled to extract second APHV data that is an average value of the first APHV data. In the exemplary embodiments, the second APHV data extraction unit 70 may be controlled to extract the second APHV data through an algorithm for calculating an average value obtained by adding all observed values together and dividing the sum by the number of observed values.

The growth age calculation unit 80 may be controlled to calculate the growth age of the measurement subject based on the second APHV data that is an average value of the first APHV data, and the predicted APHV.

In this case, the growth age calculation unit 80 may be controlled to calculate the growth age of the measurement subject using Equation 1 below.

$${GA}_{i}^{′} = ( {CA}_{i} - {APHV}_{i}^{′} ) + {APHV}_{G} \qquad [ Equation 1 ]$$

Here, GAᵢ′ may denote an estimated growth age of the measurement subject, CAᵢ may denote a chronological age/calendar age of the measurement subject, APHVᵢ′ may denote the predicted APHV data including the estimated and predicted APHV values of the measurement subject, and APHV_{G} may denote the second APHV data which is a representative value or an average value of the first APHV data derived from a training data group.

That is, in the case of a conventional growth age estimation model, generally, a growth age is calculated using a bone age, the degree of opening/closing of a growth plate, and the like, which necessarily requires X-ray measurement of the bone or the growth plate, and the growth age may be measured differently depending on a subject who analyzes X-ray photographs of the bone or the growth plate, and therefore, there are disadvantages in that objectivity is not guaranteed and accuracy is low.

On the other hand, the growth age estimation model based on biometric data using artificial intelligence according to exemplary embodiments of the present invention applies a tracking growth velocity extracted from past information to a statistical methodology to estimate a growth velocity curve, extracts an age of peak height velocity (APHV) data representing an age at which the growth velocity reaches its maximum with respect to the growth velocity curves, and then utilizes the APHV data for construction of growth age prediction models, thereby making it unnecessary to perform additional processes such as X-ray measurement, and since a correct numerical value is applied to a preset equation to determine the growth age, the growth age is less likely to be measured differently depending on the subject, thereby guaranteeing the objectivity of growth age estimation and improving accuracy.

FIG. 2 is a diagram illustrating a predicted height and target height for each growth stage according to exemplary embodiments of the present invention.

Referring to FIG. 2, in the growth stage, the childhood and adolescent period may include a normal growth period 301, a rapid growth period 303, a decelerated growth period 305, a slow growth period 307, and a growth plate closure period 309.

Each growth stage may be classified according to the degree of growth, annual growth in height differs depending on each growth stage, and actual growth in height may differ depending on a growth type even within the same growth stage.

The normal growth period 301 is a period before puberty in which secondary sexual characteristics appear, children and adolescents corresponding to this period generally have open growth plates, and in some growth environments, the height generally increases by about 4 to 5 cm per year in a small height growth type, and the height increases in a range of about 6 to 7 cm per year in a large height growth type. In exemplary embodiments, the normal growth period 301 may be defined as a period before the onset of a pubertal growth spurt in children and adolescents, and may be a growth stage ending at a point in time of the start of a first growth period preceding the predicted APHV. In one embodiment, the first growth period may be a period between one year and two years, such as about 1.5 years.

The rapid growth period 303 is a period when secondary sexual characteristics begin to appear, and in the case of females, breasts swell and breast buds form, and in the case of males, testes enlarge, pubic hair begins to grow, and a change in the voice, which is called pubertal voice change. The rapid growth period 303 generally continues for about two to three years after the normal growth period 301, and growth occurs in a range of about 7 to 10 cm per year on average. In exemplary embodiments, the rapid growth period 303 may be defined as a period in which the pubertal growth spurt begins and the growth velocity increases until APHV, and may be a growth stage starting at the point in time of the start of the first growth period preceding the predicted APHV and ending at the predicted APHV. In one embodiment, the first growth period may be a period between one year and two years, such as about 1.5 years.

The decelerated growth period 305 is a period in which secondary sexual characteristics are fully developed. In this period, in females, distinction may be made based on the onset of menarche, and in males, the change is clearly known based on pubic hair, voice change, and axillary hair. When the decelerated growth period 305 begins, a degree of growth rapidly decreases compared to the rapid growth period 303, and the decelerated growth period 305 generally lasts about two to three years, in which growth occurs at an average rate of about 5-6 cm per year, and then naturally ceases. Growth plates begin to gradually close after the rapid growth period 303, and about 50% are closed about six months after entering the decelerated growth period 305. In exemplary embodiments, the decelerated growth period 305 may be defined as a period in which the growth velocity gradually decreases until the pubertal growth spurt ends after the APHV, and may be a growth stage starting at the predicted APHV and ending at a point in time of the end of a second growth period after the predicted APHV. In one embodiment, the second growth period may be a period between one year and two years, such as about 1.5 years.

The slow growth period 307 may be a period in which the growth velocity approaches 0 before the onset of the growth plate closure period after the pubertal growth spurt. In exemplary embodiments, the slow growth period 307 may be defined as a period in which the growth velocity gradually converges to 0 after the pubertal growth spurt of children and adolescents ends, and may be a growth stage starting at the point in time of the end of the second growth period after the predicted APHV and ending at a point in time of the end of a third growth period after the predicted APHV. In one embodiment, the third growth period may be a period between two years and four years such as about three years.

The growth plate closure period 309 is a period in which the growth period has completely ended, but natural growth in height has become difficult, and is a period in which the growth plates have closed. Generally, girls enter the growth plate closure period 309 about one year six months to two years after menarche, and boys enter the growth plate closure period 309 about one year six months to two years after a point in time when axillary hair appears. In the growth plate closure period 309, growth plates are closed and natural growth stops, but growth may still occur in a range of about 1 to 3 cm by improving physical functions through a change in poor lifestyle habits, customized exercise, posture correction, and nutrient intake. In exemplary embodiments, the growth plate closure period 309 may be defined as a period in which the growth velocity is close to 0 after the growth plates are closed, and may be a growth stage starting at a point in time of the end of the third growth period after the predicted APHV. In one embodiment, the third growth period may be a period between two years and four years such as about 3 years.

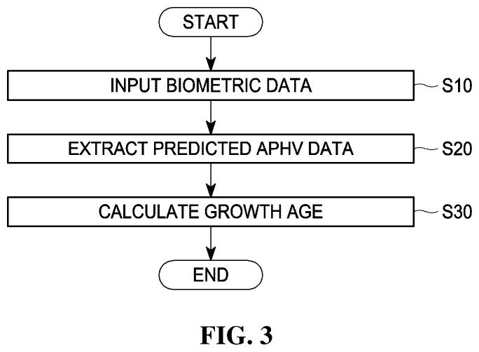

FIG. 3 is a flowchart illustrating a growth age estimation method based on biometric data using artificial intelligence according to exemplary embodiments of the present invention.

Referring to FIG. 3, the growth age estimation method using an artificial intelligence model trained with first age of peak height velocity (APHV) data representing an age at which a growth velocity of a plurality of sample subjects reaches a maximum value, as target data, may include an operation S10 of receiving biometric data of a measurement subject; an operation S20 of extracting predicted APHV data representing an age at which the growth velocity is expected to reach a maximum value, using the biometric data of the measurement subject; and an operation S30 of calculating a growth age of the measurement subject based on second APHV data that is an average value of the first APHV data, and the predicted APHV.

The artificial intelligence model trained using the first APHV data representing an age at which a growth velocity of the plurality of sample subjects reaches a maximum value as the target data will be first described, and the artificial intelligence model may be an artificial intelligence model constructed through: an operation of receiving time-series biometric data of a plurality of sample subjects; an operation of estimating a plurality of first growth velocity curves showing a velocity of height growth with respect to actual age of each of the plurality of sample subjects using the time-series biometric data of the sample subjects; an operation of extracting the first APHV data representing the age at which the growth velocity reaches a maximum value for each of the plurality of first growth velocity curves; and an operation of training the artificial intelligence model by inputting the first APHV data to the artificial intelligence model as target data.

In this case, the first APHV data is a set of data obtained by receiving the biometric data of the plurality of sample subjects, estimating a plurality of first growth velocity curves showing a velocity of height growth with respect to actual age for each of the plurality of sample subjects, and extracting an age at which the growth velocity reaches a maximum value for each of the plurality of first growth velocity curves.

Here, the operation of estimating the first growth velocity curves may be performed by extracting age data and height data from the time-series biometric data of the sample subjects and then applying the age data and the height data to a curve transformation model as input variables.

In exemplary embodiments, the curve transformation model may be a model constructed using a polynomial function.

In one embodiment, the curve transformation model may be a model constructed using Equation 2 below.

$${y}_{it} = {α}_{i} + h ( \frac{t - {β}_{i}}{exp ( - {γ}_{i} )} ) \qquad [ Equation 2 ]$$

In Equation 2, yᵢₜ is, αᵢ, βᵢ, and γᵢ are random correction values for adjusting the vertical shift, horizontal shift, and slope of the curve, respectively, h may be a specific function related to the height data, and t may be the age data.

Meanwhile, the artificial intelligence model according to the present invention may further include an operation (not shown) of preprocessing the time-series biometric data of the sample subjects, which is performed after the operation of inputting the time-series biometric data of the sample subjects.

The preprocessing operation may be performed by determining all the pieces of biometric data of the sample subject without biometric data collected during a specific growth period to be noise and removing the biometric data.

In exemplary embodiments, the preprocessing operation may be performed by classifying the growth periods of the plurality of sample subjects into a normal growth period, a rapid growth period, and a decelerated growth period and then removing as noise all pieces of data of a sample subject without biometric data corresponding to each classified growth stage.

In one embodiment, the preprocessing operation may also be performed by further classifying the growth periods of the plurality of sample subjects into a slow growth period and a growth plate closure period and then removing as noise all pieces of data of a sample subject without biometric data corresponding to each classified growth stage.

For example, the preprocessing operation may be performed by classifying the age range of the plurality of sample subjects from 8 to 18 years old and then removing as noise all pieces of data of a sample subject of which an input period difference between adjacent biometric data is 4 years or more.

Alternatively, the preprocessing operation may be performed by removing as noise all pieces of data of a sample subject without biometric data input during a period between 9 and 14 years old in the age range of the plurality of sample subjects.

The biometric data input operation S10 may be performed by receiving the physical information of the measurement subject through the biometric data input unit 10. In this case, the physical information of the measurement subject may include not only basic information such as grade (or age), gender, and height, but also the additional information such as weight, protein, mineral content, body fat, body water, muscle mass (soft lean mass), fat-free mass, bone tissue, skeletal muscle mass, body mass index (BMI), basal metabolic rate, neck circumference, chest circumference, abdominal circumference, thigh circumference, arm circumference, and hip circumference. However, such physical information is merely an example for facilitating understanding of the present invention, and the present embodiment is not limited thereto and it is obvious that the types of information constituting the physical information can vary according to embodiments.

Further, the physical information of the measurement subject may be single physical information measured once, or may be physical information measured multiple times, that is, time-series continuous information. However, the concept of the present invention is not necessarily limited thereto, and the physical information of the measurement subject may be temporally discontinuous information. That is, the physical information of the measurement subject may be collected at various collection times and collection frequencies.

In one embodiment, the biometric data of the measurement subject may include at least two or more pieces of time-series biometric data measured at different ages.

The operation S20 of extracting predicted APHV data may include an operation of estimating an expected growth velocity profile using the biometric data of the measurement subject, and an operation of extracting a predicted age of onset of growth spurt (AOGS) representing an age at which an increase in the growth velocity of the measurement subject begins, and predicted APHV representing an age at which the growth velocity of the measurement subject has a maximum value.

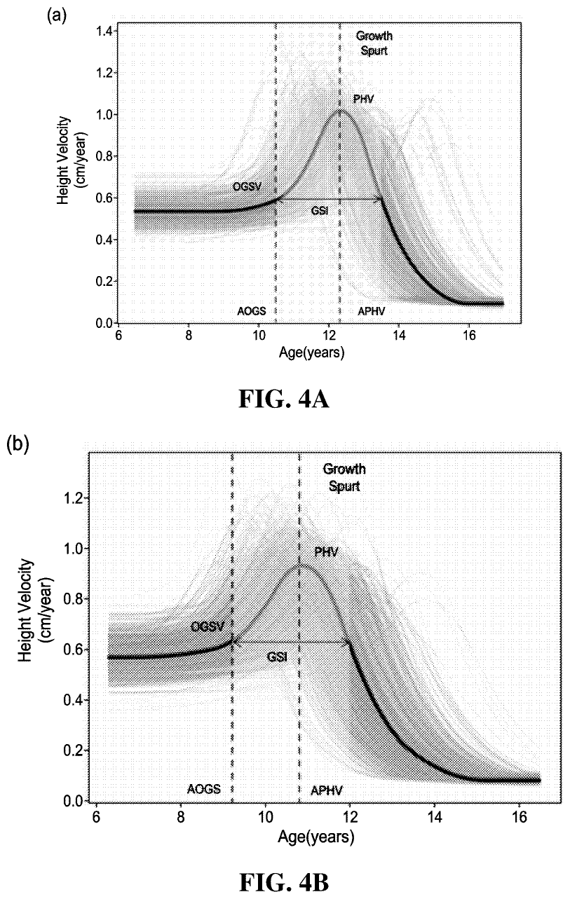

In exemplary embodiments, in the operation S20 of extracting predicted APHV data, the operation of estimating the expected growth velocity profile of the measurement subject may be performed by generating a graph (hereinafter referred to as a second growth velocity curve) indicating an expected growth velocity of the measurement subject using a single piece or a plurality of pieces of data, and then extracting predicted AOGS data indicating an expected AOGS value and predicted APHV data indicating an expected APHV value from the second growth velocity curve, as illustrated in FIGS. 4A-4B.

A case in which the operation S20 of extracting predicted APHV data is performed by generating the second growth velocity curve based on the biometric data of the measurement subject, and then extracting the predicted AOGS data and the predicted APHV data from the second growth velocity curve has been described, but the concept of the present invention is not necessarily limited thereto. That is, the operation S20 of extracting predicted APHV data may be controlled to directly extract the predicted AOGS data and the predicted APHV data through a preset algorithm without the need to generate a second growth velocity curve when the biometric data of the measurement subject is merely input to a growth age estimation model using an artificial intelligence model.

In the exemplary embodiments, the operation of estimating the first growth velocity curve may be performed by extracting the age data and the height data from the time-series biometric data of the sample subjects, and then applying the age data and the height data to the curve transformation model with the age data and the height data as input variables.

The operation S30 of calculating a growth age may be performed using Equation 1 below.

$${GA}_{i}^{′} = ( {CA}_{i} - {APHV}_{i}^{′} ) + {APHV}_{G} \qquad [ Equation 1 ]$$

Here, GAᵢ′ may denote an estimated growth age of the measurement subject, CAᵢ may denote a chronological age/calendar age of the measurement subject, APHVᵢ′ may denote the predicted APHV data including the estimated and predicted APHV values of the measurement subject, and APHV_{G} may denote the second APHV data which is a representative value or an average value of the first APHV data derived from a training data group.

That is, a growth age (Ega) of the measurement subject calculated through the operation S30 of calculating a growth age may be calculated by correcting second APHV data (S_{AA}), that is, an average value of the first APHV data by a difference between an actual age (Eᵣₐ) of the measurement subject and a predicted APHV data (E_{PA}) of the measurement subject.

Therefore, since all of the actual age of the measurement subject, that is, the actual chronological age uniformly calculated from the time of birth, the predicted APHV data calculated using the tracking growth velocity of the measurement subject, and the second APHV data corresponding to a group average APHV of the sample subjects can be applied to estimate a biological growth age of the measurement subject, and the biological growth age can be utilized for construction of various prediction models such as an integrated height growth prediction model, a precocious puberty prediction model, and an obesity prediction model, it is possible to maximize the reliability of various prediction models such as the integrated height growth prediction model, the precocious puberty prediction model, and the obesity prediction model.

FIGS. 4A-4B are diagrams illustrating a plurality of first growth velocity curves showing a velocity of height growth with respect to actual age using the time-series biometric data of the sample subjects.

In FIGS. 4A-4B, a plurality of first growth velocity curves generated through a first growth velocity curve estimation unit 30 are illustrated, and the first APHV data extraction unit 40 may be controlled to extract the first APHV data representing the age at which the growth velocity reaches a maximum value for each of the plurality of first growth velocity curves. The first APHV data may be data indicating an age at which the growth velocity reaches its maximum during the pubertal growth spurt, that is, growth acceleration is zero.

Meanwhile, the first APHV data extraction unit 40 may also be controlled to extract additional data other than the first APHV data representing the age at which the growth velocity reaches a maximum value for each of the plurality of first growth velocity curves.

For example, the first APHV data extraction unit 40 may be controlled to additionally extract age of onset of growth spurt (AOGS) data representing a point at which the pubertal growth spurt begins, and age of end of growth spurt (AEGS) data representing a point at which the pubertal growth spurt ends.

The AOGS data may mean a point at which the growth velocity slope rapidly increases and growth acceleration reaches its maximum, and may represent data indicating an age about 1.5 years before the first APHV data.

The AEGS data may represent the point at which the pubertal growth spurt ends, that is, a point at which the growth velocity after APHV becomes equal to the growth velocity at AOGS (onset of growth spurt velocity), and may be data indicating an age about 1.5 years after the first APHV data.

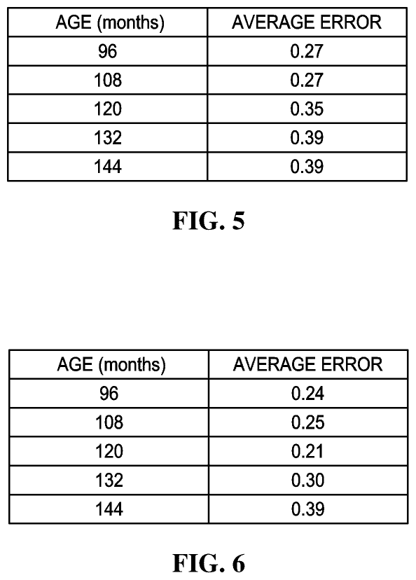

FIGS. 5 and 6 are diagrams illustrating APHV estimation errors according to age in months for girls and boys, respectively.

Referring to FIGS. 5 and 6, predicted APHV data of measurement subjects of various ages was extracted using the APHV estimation model based on biometric data using artificial intelligence according to exemplary embodiments of the present invention, and then the predicted APHV data was compared with the actual APHV data of the measurement subjects.

Specifically, it was confirmed from such experiment results that, for girls aged 10 years or older, an average error between a predicted APHV data value and an actual APHV data value was 0.35 years, and for boys aged 12 years or older, the average error between the predicted APHV data value and the actual APHV data value was 0.39 years. That is, as a result, is was confirmed that, in the case of the APHV estimation model based on biometric data using artificial intelligence according to exemplary embodiments of the present invention, for both girls and boys, the average error between the predicted data value and the actual data value at an average age at which secondary sexual characteristics appear was within 0.5 years, and the accuracy of APHV prediction was very high.

Further, it was confirmed from such experiments that the prediction accuracy of the APHV estimation model is improved as the actual ages of the measurement subjects are closer to the actual APHV data value.

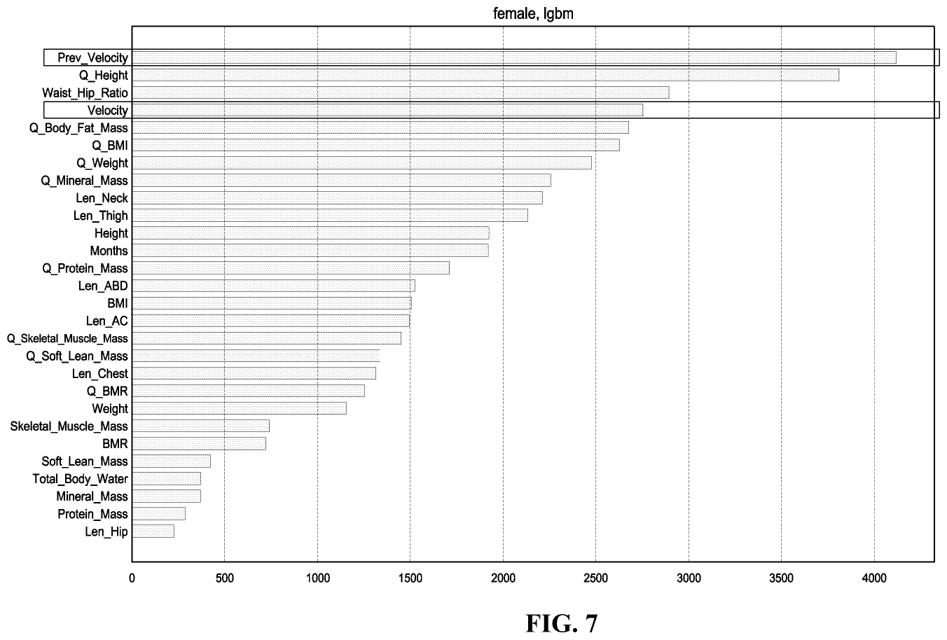

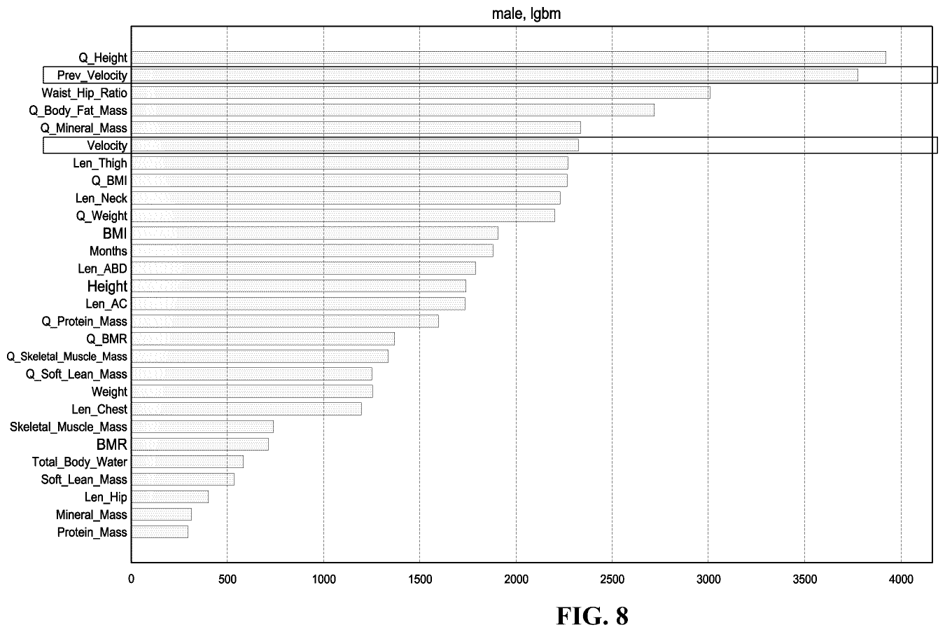

FIGS. 7 and 8 are diagrams illustrating the importance of APHV prediction variables for females and males, respectively.

Referring to FIGS. 7 and 8, various variables for constructing the APHV estimation model based on biometric data using artificial intelligence according to exemplary embodiments of the present invention were classified depending on their importance.

Specifically, as illustrated in FIG. 7, in prediction of the female APHV, a variable with the highest importance was revealed to a previous growth velocity, whereas as illustrated in FIG. 8, in prediction of the male APHV, the variable with the highest importance was revealed to be a height quantile.

Meanwhile, in the prediction of the female APHV, a current growth velocity was revealed to be a variable with the fourth most importance, and in the prediction of the male APHV, the previous growth velocity and current growth velocity were revealed to be variables with the second and sixth most importance, respectively.

That is, it was confirmed that a previous growth velocity and a current growth velocity each serve as very important variables for prediction of the APHV in both females and males, and in particular, the previous growth velocity was revealed to be the most important variable in the case of females and to be a variable with second most importance in the case of males. As a result, it was confirmed that the accuracy of the predicted APHV data extracted using the APHV estimation model can be greatly improved when the previous growth velocity can be confirmed due to the presence of two or more pieces of biometric data measured for the measurement subject at different points in time.

Further, it was confirmed that, in order to increase the accuracy of the predicted APHV data extracted through the APHV estimation model based on biometric data using artificial intelligence according to exemplary embodiments of the present invention, additional variables such as height quantile, waist to hip ratio (WHR), and body fat mass are required.

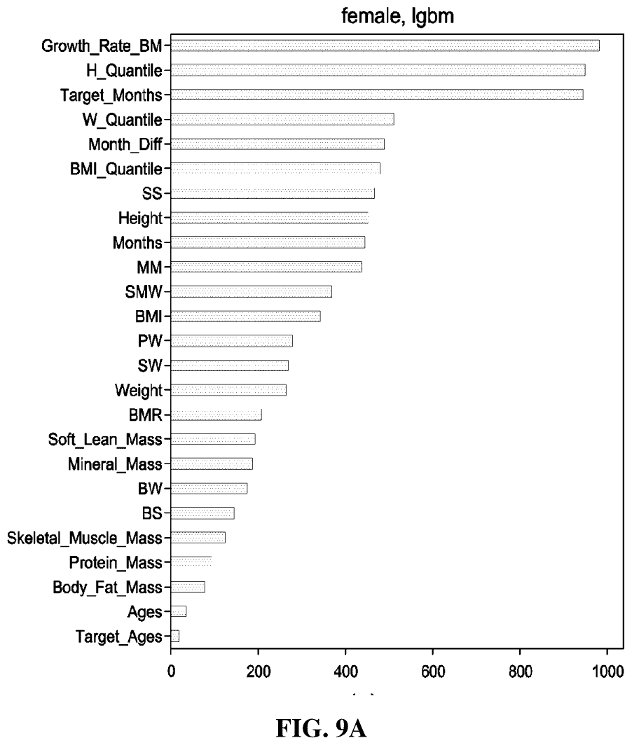

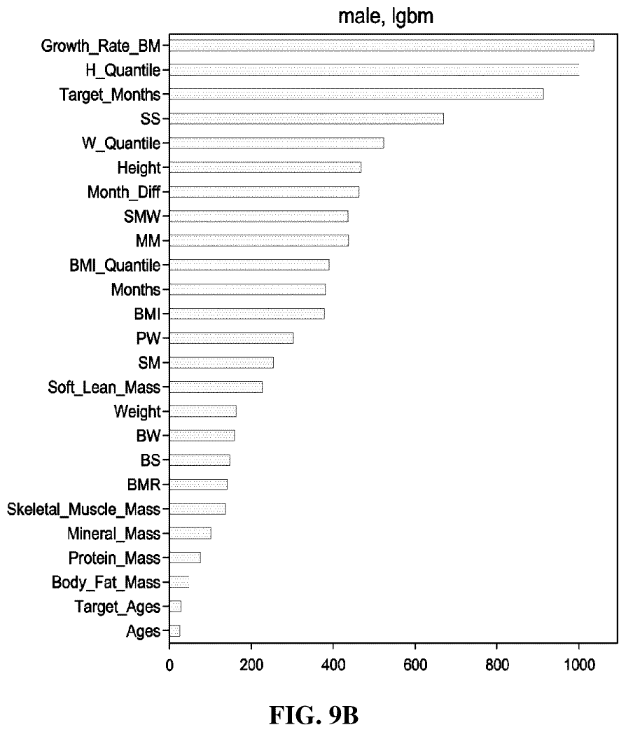

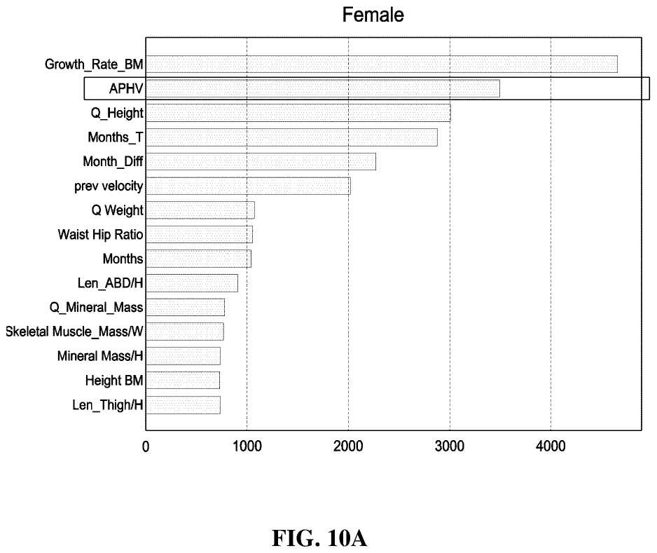

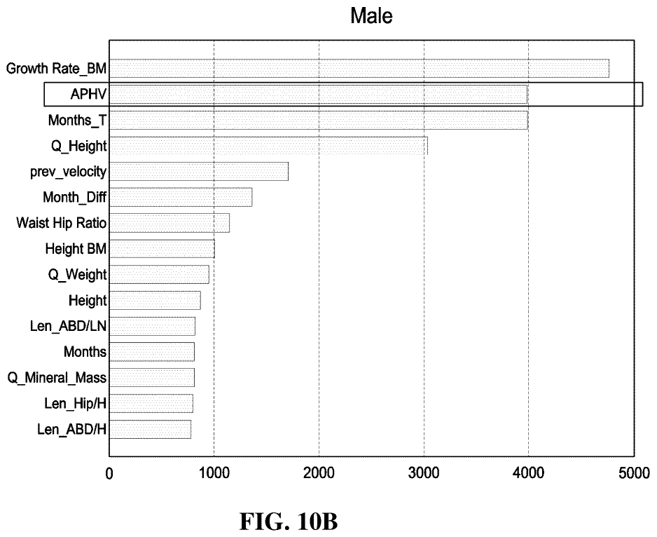

FIGS. 9A-9B are diagrams illustrating variable importance in conventional female and male growth prediction models, and FIGS. 10A-10B are diagrams illustrating variable importance in the growth prediction model of the present invention.

Referring to FIGS. 9 and 10, in both the conventional female and male growth prediction models and the growth prediction model of the present invention, the variable with the highest importance was revealed to be a growth rate, and in the conventional growth prediction model, the variable with second most importance was revealed to be the height quantile, whereas in the growth prediction model of the present invention, the variable with second most importance was revealed to be the APHV.

That is, in the conventional growth prediction model prior to application of the APHV concept, the height quantile was revealed to be the variable with second most importance after the growth rate, whereas in the growth prediction model according to the present invention to which the APHV concept is applied, the APHV was calculated as having higher importance than the height quantile and was revealed to be the variable with second most importance after the growth rate, and therefore, it was confirmed that the APHV has very high importance among variables affecting predicted final height.

As described above, the APHV estimation model based on biometric data using artificial intelligence according to exemplary embodiments of the present invention applies a tracking growth velocity extracted from past information to a statistical methodology to estimate a growth velocity curve, extracts APHV data representing an age at which the growth velocity reaches its maximum with respect to the growth velocity curves, and then utilizes the APHV data for construction of various prediction models such as an integrated height growth prediction model, a precocious puberty prediction model, and an obesity prediction model, thereby maximizing the reliability of various prediction models such as the integrated height growth prediction model, the precocious puberty prediction model, and the obesity prediction model.

Further, according to various embodiments of the present invention, it is possible to accurately predict the heights of children and adolescents in consideration of the growth stages of children and adolescents using the APHV estimation model constructed through artificial intelligence learning, and to provide a solution necessary for growth in height in consideration of each growth stage.

However, the concept of the present invention is not necessarily limited thereto, and the apparatus, method, and system according to exemplary embodiments of the present invention can also be applied to various products and technical fields other than the above-described ones.

Although various embodiments of the present invention have been described above in detail, those skilled in the art will understand that various modifications can be made to the above-described embodiments without departing from the scope of the present invention. Therefore, the scope of rights of the present invention should not be limited to the described embodiments and should be defined by the claims to be described below and equivalents thereof.

1. A growth age estimation method using an artificial intelligence model trained with first age of peak height velocity (APHV) data representing an age at which a growth velocity of a plurality of sample subjects reaches a maximum value, as target data, the method comprising:
receiving biometric data of a measurement subject;
extracting predicted APHV data representing an age at which the growth velocity is expected to reach a maximum value using the biometric data of the measurement subject; and
calculating a growth age of the measurement subject based on second APHV data that is an average value of the first APHV data, and the predicted APHV.

2. The growth age estimation method of claim 1, wherein the calculating of the growth age is performed using Equation 1:
$${GA}_{i}^{′} = ( {CA}_{i} - {APHV}_{i}^{′} ) + {APHV}_{G} \qquad [ Equation 1 ]$$
where GAᵢ′ denotes an estimated growth age of the measurement subject, CAᵢ denotes a chronological age/calendar age of the measurement subject, APHVᵢ′ denotes the predicted APHV data including the estimated and predicted APHV values of the measurement subject, and APHV_{G} denotes the second APHV data which is a representative value or an average value of the first APHV data derived from a training data group.

3. The growth age estimation method of claim 1, wherein the extracting of the predicted APHV data includes:
estimating an expected profile of the growth velocity using the biometric data of the measurement subject; and
extracting a predicted age of onset of growth spurt (AOGS) representing an age at which an increase in the growth velocity of the measurement subject begins, and a predicted APHV representing an age at which the growth velocity of the measurement subject has a maximum value.

4. The growth age estimation method of claim 1, wherein the biometric data of the measurement subject includes at least two or more pieces of time-series biometric data measured at different ages.

5. The growth age estimation method of claim 1, wherein the first APHV data is a set of data obtained by receiving biometric data of the plurality of sample subjects, estimating a plurality of first growth velocity curves showing a velocity of height growth with respect to actual age for each of the plurality of sample subjects, and extracting an age at which the growth velocity reaches a maximum value for each of the plurality of first growth velocity curves.

6. A growth age estimation method using an artificial intelligence model trained with parameters and feature points for determining a plurality of first growth velocity curves showing a velocity of height growth with respect to actual age of a plurality of sample subjects as target data, the method comprising:
receiving biometric data of a measurement subject;
extracting predicted age of peak height velocity (APHV) data representing an age at which the growth velocity is expected to reach a maximum value using the biometric data of the measurement subject; and
calculating the growth age of the measurement subject based on the predicted APHV and second APHV data that is an average value of first APHV data representing an age at which a growth velocity of the plurality of sample subjects reaches a maximum value among feature points determining the plurality of first growth velocity curves.

7. The growth age estimation method of claim 6, wherein the calculating of the growth age is performed using Equation 1:
$${GA}_{i}^{′} = ( {CA}_{i} - {APHV}_{i}^{′} ) + {APHV}_{G} \qquad [ Equation 1 ]$$
where, GAᵢ′ denotes an estimated growth age of the measurement subject, CAᵢ denotes a chronological age/calendar age of the measurement subject, APHVᵢ′ denotes the predicted APHV data including the estimated and predicted APHV values of the measurement subject, and APHV_{G} denotes the second APHV data which is a representative value or an average value of the first APHV data derived from a training data group.

8. The growth age estimation method of claim 6, wherein the biometric data of the measurement subject includes at least two or more pieces of time-series biometric data measured at different ages.

9. The growth age estimation method of claim 6, wherein the first APHV data is a set of data obtained by extracting an age at which the growth velocity reaches a maximum value for a first growth velocity curve of each of the plurality of sample subjects.

10. The growth age estimation method of claim 9, wherein the first growth velocity curve is estimated by extracting age data and height data among the time-series biometric data of the sample subjects and then applying the age data and the height data to a curve transformation model with the age data and the height data as input variables.

11. The growth age estimation method of claim 8, further comprising:
preprocessing the time-series biometric data of the sample subjects, which is performed after the receiving of the time-series biometric data of the sample subjects,
wherein the preprocessing is performed by determining all the pieces of biometric data collected during a specific growth period falling outside a preset range to be noise and removing the biometric data.

12. The growth age estimation method of claim 11, wherein the preprocessing is performed by classifying the growth periods of the plurality of sample subjects into a normal growth period, a rapid growth period, and a decelerated growth period and then removing as noise all pieces of biometric data corresponding to each classified growth stage falling outside a preset range.

13. The growth age estimation method of claim 12, wherein the preprocessing is performed by further classifying the growth periods of the plurality of sample subjects into a slow growth period and a growth plate closure period and then removing as noise all pieces of biometric data corresponding to each classified growth stage falling outside a preset range.

14. The growth age estimation method of claim 13,
wherein the slow growth period is defined as a period in which the growth velocity gradually converges to 0 after a pubertal growth spurt of children and adolescents ends, and
the slow growth period is a growth stage starting at a point in time of the end of a second growth period after the predicted APHV and ending at a point in time of the end of a third growth period after the predicted APHV.

15. The growth age estimation method of claim 14, wherein the second growth period is a period between one year and two years, and the third growth period is a period between two years and four years.
